(19) United States
(12) Patent Application Publication
(10) Pub. No.: US 2010/0280505 A1
(43) Pub. Date: Nov. 4, 2010

(54) Device for Localized Thermal Ablation of Biological Tissue, Particularly Tumoral Tissues or the Like

(75) Inventors: Marco MATTIUZZI, Grassina (Bagno a Ripoli), Firenze (IT); Gaetano CASCINI, Firenze (IT); Davide RUSSO, Firenze (IT)

Correspondence Address: Themis Law, 7979 Ivanhoe Ave Ste 400, La Jolla, CA, 92037, US

(73) Assignee: BRACCO IMAGING S.P.A., Milano (IT)

(21) Appl. No.: 12/305,973

(22) PCT Filed: Jun. 28, 2007
(86) PCT No.: PCT/EP2007/056508

§ 371 Date: Feb. 21, 2009

(30) Foreign Application Priority Data
Jul. 4, 2006 (EP) 06425459.2

(51) Int. Cl. A61B 18/22

(52) U.S. Cl. 606/15

(57) ABSTRACT

Device for localized thermal ablation of lesion tissues, particularly tumoral tissues or the like, which device comprises: a probe or needle intended to be positioned with the end tip at the lesion tissue or tumoral tissue area to be removed; which probe or needle support at least a light guide as an elongated member like a thin wire or thread, one of the ends thereof is an end emitting heating electromagnetic energy and which light guide ends at said end of the probe or needle by a tip irradiating said electromagnetic energy, particularly as a laser light and the other end thereof is connected to a source generating the electromagnetic energy; means for controlling the activation/deactivation of the source generating the electromagnetic energy. Characterized in that it comprises in combination means for controlled distribution of the heating action on the lesion tissue area generated by the electromagnetic energy emitted by the irradiating tip inside a volume having a predetermined size, which means are composed of a fluid or a mixture or a combination of heat storing and/or conveying fluids or substances there being provided means for distributing, conveying and/or containing said fluid or fluids or said substance or substances inside the area to be treated or for conveying or containing said fluid or fluids or said substance or substances outside the area to be treated.

The present invention relates to a device for localized thermal ablation of lesion tissues, particularly tumoral tissues or the like, which device comprises:

a probe or needle intended to be positioned with the end tip at the lesion tissue or tumoral tissue area to be removed;

which probe or needle support at least a light guide as an elongated member like a thin wire or thread, one of the ends thereof is an end emitting heating electromagnetic energy and which light guide ends at said end of the probe or needle by a tip irradiating said electromagnetic energy, particularly as a laser light and the other end thereof is connected to a source generating the electromagnetic energy;

means for controlling the activation/deactivation of the source generating the electromagnetic energy.

and means for distributing the heating action on the lesion tissue area generated by the electromagnetic energy emitted by the irradiating tip inside a volume having a predetermined size, which means are composed of a fluid or a mixture or a combination of heat storing and/or conveying fluids or substances

These type of devices are known and are used in medical field particularly for ablating tumoral tissues. The great advantage of these devices is that the ablating action is little invasive and periods in bed of the patient are very short.

Document US-A-2002177846 discloses a system for carrying out termoablation of tissues using vapour. The said vapour is generated outside the body and then it is fed by means of a cannulain the area of the tissue to be treated inside the body. The steam can also be generated at the location of the tissue to be treated by means of a cannula. Means for measuring the temperature are alos provided. The heat is transferred to the tissue to be treated directly through bthe vapour or indirectly by heating at the site of the tissue to be treated of a fluid, particularly a liquid. Thus in both cases a fluid must be provided either outside the body for generating the vapour or inside the body at the location of the tissue to be treated for being heated.

Document U.S. Pat. No. 5,944,713 discloses a system fo the termoablation of lesioned tissues, particularly for void organs. The system uses a cannula inside of which optical fibres are provided for emitting electromagnetic energy. This energy is used for heating a fluid which serves as a transmission mean for the heat generated by the elctromagentic energy to the tissue to be treated.

Document EP-A-0 292 622 discloses a particular kind of needle for medical treatments among which also the termoablation is considered. The needle comprises inside it a laser light emitter. In the vicinity of the emission tip, thanks to a particular shaping of the said tip a special substance, in particular manganese dioxide is fed there. The electromagnetic energy which is emitted heats directly the tissue to be treated and at the same time it heats also the manganese dioxide which afterwards emits the absorbed heat to the tissue to be treated, working as a heat transfer media.

Document US-A-2002015159 relates to a method of termoablation which is based on the use of ferromagentic particles. The said particles are distributes in such a way as to permeate the area tio be treated and afterwards the particles are irradiated wiht an external magnetic ield of predetermined intensity. Due to the said magnetic field, the ferromagentic particles are heated by beibg excited by the said field and thus the said particles work as heat emitter for transferring the heat to the tissues. The said particles are lined with bicompatible polimers in order not to be dangerous for the patient.

None of the above disclosed systems or methods seems to provide means for controlling the heat transfer. The media used for transferring the heat are simply heated and brought into contact with the tissue to be treated.

At present various studies have been made showing a very good result of these devices for removing small tumoral masses with a diameter of about 10 mm. Instead as regards greater tumoral masses, the treatment thereof is still difficult and however the complete removal of the tumoral tissue requires the movement of the probe and other expedients.

The light guide is generally composed of a filament of optical fiber with the great advantage of having a reduced attenuation of conveyed luminous energy and allowing to restrict the invasive effect of the needle or probe by the fact that the needle or probe wherein the end portion of the light guide is inserted can have very small diameters. On the other hand that is advantegous considering the fact that some tumoral lesions may be in anatomical districts crowded with organs having functions also of the vital type so the insertion of the probe or needle is related to relatively high damage elements.

The light source is generally composed of a laser light source suitable to provide the necessary intensity to increase temperature of irradiated tissue up to levels necessary for the treatment.

However the practical use of thermal ablation by devices irradiating electromagnetic energy is still subjected to further problems especially occurring when treating very large lesion tissue areas. In this case, the problem of treating a large lesion can be considered to be solved simply by a marking of the tip of the probe or needle for example by various imaging means like ultrasound ones or the like and so by moving the needle or probe with respect to the tissue area to be treated to such an extent necessary to treat said tissue area along its whole size. However in this case there is the problem of moving the needle or probe with respect to the area to be treated and so by orienting the tip irradiating the electromagnetic energy in order to irradiate a different portion of said area only when the area currently subjected to irradiation has been completely treated. Therefore there is the problem of determining when a partial area belonging to a larger area of lesion tissue has been treated in a way sufficient to determine desidered therapeutic effects. In addition to this problem there is also a further problem that has been found in such thermal ablation devices operating with irradiation of electromagnetic energy and it is the fact that water vapour is generated due to tissue heating. The presence of vapour makes impossible to use a simple and inexpensive technique such as ultrasound images for monitoring the treatment. Ultrasounds are just blinded with the presence of vapour.

From a more general point of view, as regards thermal ablation by irradiating with laser light the lesion tissue there is the problem of obtaining an even distribution of the heating effect on a large area or anyway on an area or volume that are larger than the tissue area directly exposed to the output end of the guide fiber of the light ray, so called fiber tip or tip. The area directly adjacent to the output end of the guide fiber of the laser beam is a small area or very small volume so the heating action is very deep in this small area whereas the heating action quickly decreases as the distance from said output end increases. Therefore there is the risk of exerting an excessive heating action in the areas directly adjacent to the tip, and of treating more distant areas in an insufficient way. Therefore it is necessary to have a means for learning or controlling the real spatial distribution of the heat or heating effect in the volume surrounding the tip and depending on the distance from the tip in order to control the heating effect and therefore the desired treatment.

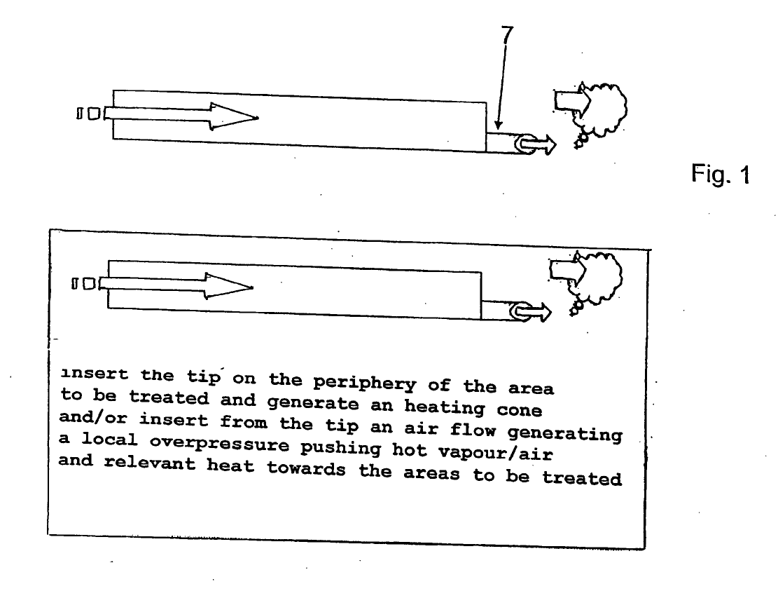

Various studies have been made in order to understand interactions between tissues and electromagnetic energy, particularly as laser beams. FIG. 1 very schematically shows how the energy of laser irradiation on a tissue in the process called laser volatilization of tissues is considered to work. It is a process used for incision and tumoral ablation. During tissue removal three steps have been pointed out which have been defined as follows even referring to the temperature range taken by the tissue: coagulation defines the tissue heating condition at temperatures between 55-100° C.: water vaporization defines the heating step between 100 and 400° C.; combustion occurs when heating exceeds 400° C. It has been found that tissue mass loss is due firstly to radiating flux of the treated area. As regards fluxes with values exceeding 1000 J/cm², the pointed out effect corresponds to a whitening of the tissue. The phenomenon so called of popcorn vaporization occurs at thermal energy fluxes between 1100 and 1500 J/cm², while the carbonization and combustion occurs when the radiating flux exceeds 1500 J/cm².

Very in-depth studies have been made in order to determine how the heat is distributed in tissues and particularly in interfaces between healthy tissues and lesion tissues. Essentially studies have found that parameters determining the distributing of heat are complex and that it is not possible to generalize or isolate a general law that can be adapted to all conditions even approximately. Particularly the way according to which thermal diffusion works in tissues after irradiation by laser light depends not only on the radiating flux but also on the tissue quality referring above all to absorption of the electromagnetic irradiation by tissues. In this case, each different type or kind of tissue has a different behaviour and so it is diffcult to determine a priori a general law.

As regards vapour production at the moment the problem is not dealt with except by trying to modulate the energy supply, in order to have the greatest heating effect on tissue without generating vapour, by using the different response to irradiation of different tissue components, water among thereof. A solution adopted to avoid vapour formation is the alternative operation of laser source and so irradiating tissues by laser light pulses. However the solution is not a satisfactory one since the thermal ablation effect however is small with respect to what could be obtained by a constant and adjusted irradiation.

Therefore the invention aims at improving known thermal ablation devices of the type described hereinbefore allowing firstly to overcome drawbacks of known methods and that is the fact of allowing in a substantially simple and safe way to treat relatively large lesion tissue areas without the risk of burning some partial areas or of heating in an insufficient way other partial areas and at the same time allowing to treat the whole size of the lesion tissue as safely as possible.

A further aim is the fact of allowing what has been mentioned above by automatic or nearly automatic means reducing as much as possible a direct controlling intervention by the operator and allowing to standardize the ablation process.

The invention achieves the above aims by providing a device of the type described hereinbefore wherein there are provided means for controlling the heat transfer from the fluid to the tissue by conveying and/or distributing a certain amount of fluid along diferent zones of the area to be treated and/or containing said fluid or fluids or said substance or substances inside different zones or the entire area to be treated or for conveying or containing said fluid or fluids or said substance or substances outside the area to be treated.

According to an embodiment of the invention means for distributing the heat are means distributing the heat generated by the electromagnetic irradiation on distributing means.

In one embodiment the device comprises means for injecting a heat storing/thermoregulating substance, particularly a substance having a predetermined temperature of the change of state as for example from liquid to gaseous and/or from solid to liquid or vice versa and which temperature corresponds to the thermal treatment temperature of the lesion. In combination with said substance there can be provided means for containing and/or retaining said substance inside a predetermined volume and/or outside it, particularly about a predetermined volume, which volume approximately coincides with the volume wherein the lesion tissue to be subjected to thermal ablation treatment is provided.

In this case the containing action of the thermoregulating substance can be obtained by a ferromagnetic behaviour thereof or by associating said substance to a conveying carrier composed of a substance with ferromagnetic properties. It is therefore possible also to provide means for generating a localized magnetic field having such a spatial position and size to permeate only the lesion tissue area and/or to surround the lesion tissue area to be treated, in order to distribute the thermoregulating substance in the volume corresponding approximately to the tissue area to be treated or around it along the surface envelopping said tissue area to be treated.

Referring to this last variant it is particularly advantageous when the thermoregulating/storing substance is a substance that works as a barrier of the heat propagation outside the lesion tissue area to be treated, the generated magnetic field being such that the ferromagnetic carrier concentrates the thermoregulating substance in an envelopping jacket of said lesion tissue area to be treated and the thermoregulating substance being provided with a vaporization or fusion temperature of 35 to 38° C.

The thermoregulating substance and/or the substance with ferromagnetic properties can be also contained in micro-bubbles or micro-balls and/or micro-bubbles or micro-balls can be the thermoregulating substance and/or the ferromagnetic substance.

A further variant provides that by means of injecting means a heat storing fluid is locally provided, particularly a substance having a predetermined temperature of the change of state as from liquid to gaseous and which temperature corresponds to the thermal treatment temperature of the lesion, which injector comes out at the output emitting the electromagnetic beam of the irradiating tip there being provided means for mechanically pushing said fluid.

The mechanical pushing of the heat storing fluid can be obtained in various ways, for example means for pushing said fluid can be composed of a direct conveying carrier composed of the natural lymphatic or vascular flow.

As an alternative to or in combination means pushing said fluid can be composed of a fluid jet there being provided on the tip of the probe or needle at least a nozzle supplying said jet or said jets.

Again as an alternative to or in combination means for pushing said fluid can be also composed of the mechanical pressure wave generated by a source of acustic waves particularly ultrasound ones. In this case low frequency ultrasound waves and with triangular or sawtooth pulse arrangement are advantageous.

A particular embodiment provides as the thermal storing fluid for transporting the thermal energy the vapour generated by heating the tissue by the electromagnetic beam coming from the irradiating tip.

Again a possible variant embodiment as regards means for controlling the thermal diffusion comprises means for controlling the vascular and/or lymphatic circulation in the area corresponding to the lesion tissue.

In such variant means for controlling the vascular or lymphatic circulation are advantageously composed of magnetorheological substances there being provided means generating localized magnetic fields operating magnetorheological substances to make agglomerates for locally preventing vascular and/or lymphatic flow generating a barrier to thermal diffusion by perfusion.

A variant can be composed of means locally coagulating the blood in the lesion tissue area.

In this case an advantageous change of functions of thermal transfer from irradiating tip of the probe or needle in the direction moving away therefrom is obtained.

The control of distribution of the heating effect can also occur by detecting physical parameters of the lesion tissue changing according to temperature of the lesion tissue, such to check the occurred treatment of portions of a very large lesion tissue area. In this case the measurement can be provided in combination with automatic means moving the needle or probe for treating a different portion conveying and/or with the operation of one or more means already described above referring to one or more different variants.

In this case the device according to the present invention comprises at least a sensor measuring physical parameters of the lesion tissue depending on heating temperature thereof, which sensor is supported at a certain distance from the irradiating tip in a predetermined position with respect thereto and which sensor measures the change of said physical parameter of the lesion tissue comprised between said irradiating tip and the sensor and means for processing the measurement signal of the sensor which determine the heating temperature of said lesion tissue area on the basis of said measurement signal, as well as signalling means and/or possibly also automatic means for modulating the electromagnetic beam and/or automatic means for moving the irradiating tip operated on the basis of said measurement signal.

Alternatively or in combination the sensor can be of the electric, temperature, acustic, optical, laser, chemical, electrochemical, luminescence, RF wave change, pH, position, micro-movement, selective-tissue type.

For a predetermined type of lesion tissue in a predetermined anatomical district the correlation function between heating effect, heat diffusion and change in the physical parameter to be measured is determined, said function being sampled and stored in a table comparing and evaluating signals generated by the sensor measuring said physical parameter.

It is to be noted how the different embodiment variants and the different ways of making them described above can be provided in any combination or subcombination one with the other when they are technically compatible in order to make the control of the heating distribution on all the size of the lesion to be removed as an abounding one and a more safe one.

The invention relates also to a method for localized thermal ablation of lesion tissues, particularly tumoral tissues or the like, which method comprises the following steps:

Generating an electromagnetic irradiation having a predetermined energy and frequency;

Irradiating locally and for a predetermined period of time, with said electromagnetic irradiation, a lesion tissure area or a portion thereof in order to increase the temperature of the lesion tissue of said area or portion thereof up to a predetermined value;

Characterized in that in combination it comprises steps controlling in an active way the distribution on the lesion tissue area of the heating action generated by the electromagnetic energy inside a volume having a predetermined size.

The control of the heat distribution is obtained by perfusing a heat storing/thermoregulating substance in the lesion tissue area, particularly a substance having a predetermined temperature of the change of state for example from liquid to gaseous and/or from solid to liquid or vice versa and which temperature corresponds to the temperature for thermally treating the lesion.

In this variant the control of the heat distribution is obtained by perfusing outside and about the lesion tissue area a heat termoregulating/storing substance, particularly a substance having a predetermined change of state temperature for example from liquid to gaseous and/or from solid to liquid or vice-versa and which temperature corresponds to the average temperature of healthy tissue about the lesion tissue area.

In this case the localized containing or retaining action inside the lesion tissue area or about the latter is obtained by ferromagnetic or storing substances having ferromagnetic properties or combined with carriers having ferromagnetic properties and by generating a localized magnetic field having such a spatial position and size to permeate only the lesion tissue area and/or to surround the lesion tissue area to be treated, in order to distribute the thermoregulating substance in the volume corresponding approximately to the tissue area to be treated or around it along the surface envelopping said tissue area to be treated.

Advantageously the thermoregulating substance can be a substance that works as a barrier of the heat propagation outside the lesion tissue area to be treated, the generated magnetic field being such that the ferromagnetic carrier concentrates the thermoregulating substance in an envelopping jacket of said lesion tissue area to be treated and the thermoregulating substance being provided with a vaporization or fusion temperature of 35 to 38° C.

Still another variant of the method provides to heat a heat storing fluid by the electromagnetic irradiation, particularly a substance having a predetermined temperature of the change of state such as from liquid to gaseous and which temperature corresponds to the temperature for thermally treating the lesion, means for pushing said fluid in the lesion tissue area being provided.

The control of the thermal diffusion may be carried out also by controlling the vascular and/or lymphatic circulation in the area corresponding to the lesion tissue.

In this case, the control of the vascular or lymphatic circulation is obtained by activating magnetorheological substances by means of localized magnetic fields to make agglomerates for locally preventing vascular and/or lymphatic flow generating a barrier to thermal diffusion by perfusion.

Still a variant of the method provides to measure physical parameters of the lesion tissue depending on the heating temperature thereof, which measurement occurs in a predetermined position with respect to a certain distance from an irradiating tip, the change of said physical parameter of the lesion tissue comprised between said irradiating tip and the measurement point being measured, while the measurement signal is processed for determining the heating temperature of said lesion tissue area on the basis of said measurement signal, as well as for generating a signaling and/or possibly for automatically controlling the modulation of the electromagnetic beam and/or the movement of the irradiating tip on the basis of said measurement signal.

Alternatively or in combination it is possible to measure an electric, temperature, acustic, optical, laser, chemical, electrochemical, luminescence, RF wave change, pH, position, micro-movement, selective-tissue parameter.

Further improvements of the device and of the method according to the present invention are object of subclaims.

Characteristics of the present invention and advantages deriving therefrom will be clear by the following description of some not limitative embodiments shown in annexed drawings wherein:

FIG. 1 is one embodiment of the invention wherein a thermal ablation probe or needle are provided with an injector for a heat distributing thermoregulating substance.

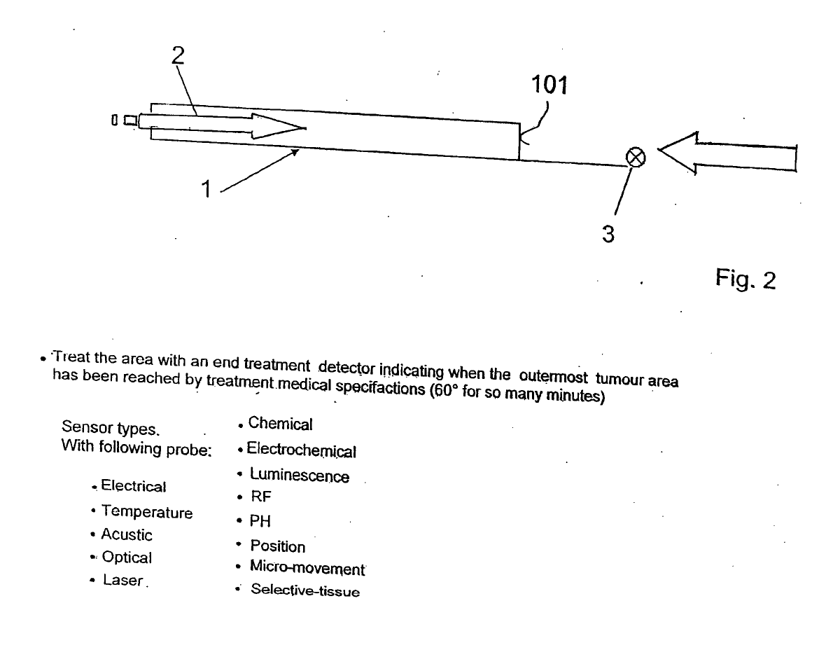

FIG. 2 is a first embodiment of the invention providing means for detecting end treatment conditions on an area or volume corresponding to the area or volume of the lesion tissue area to be treated.

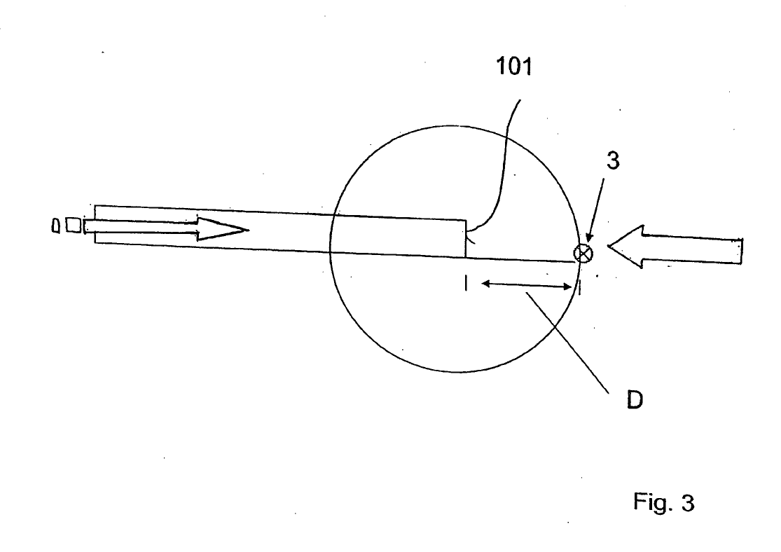

FIG. 3 is the treatment area that can be obtained by a device according to FIG. 2.

Probes or needles for thermal ablating lesion tissues, particularly tumoral tissues by heating with a laser light are known per se. Particularly current methods and devices for thermal ablation by heating with laser light are described for example in following documents: “Low Power Interstitial Photocoagulation in rat Liver, Proc. of SPIE Vol. 1882, Laser-Tissue Interaction IV, ed S. L. Jacques, A. Katzir 8 Luglio 1993) Copyright SPIE”, and U.S. Pat. No. 4,592,353, U.S. Pat. No. 4,692,244, U.S. Pat. No. 4,736,743.

FIG. 1 shows an embodiment of the invention wherein means for distributing the heat generated by the electromagnetic irradiation are composed of substances intended to be distributed or diffused or they permeate in time the tissue area to be treated and which substances are heated by the laser irradiation and distribute the heat by perfusing the tissue to be treated. In this figure the irradiating tip of the needle comprises a nozzle 7 for localizely injecting a substance intended to distribute the heat or to adjust or to make even the heating action.

According to a first variant injection means provide the injection of a heat storing/thermoregulating substance, particularly a substance having a predetermined temperature of the change of state for example from liquid to gaseous and/or from solid to liquid or vice-versa and which temperature corresponds to the thermal treatment temperature of the lesion. In combination with said substance means for containing and/or retaining said substance in a predetermined volume and/or outside it, particularly around a predetermined volume can be provided, which volume approximately coincides with the volume wherein the lesion tissue to be treated by thermal ablation is provided.

In this case the containing action of the thermoregulating substance can be obtained by a ferromagnetic behaviour thereof or by associating said substance to a conveying carrier composed of a substance with ferromagnetic properties. It is therefore possible also to provide means for generating a localized magnetic field having such a spatial position and size to permeate only the lesion tissue area and/or to surround the lesion tissue area to be treated, in order to distribute the thermoregulating substance in the volume corresponding approximately to the tissue area to be treated or around it along the surface envelopping said tissue area to be treated.

Referring to this last variant it is particularly advantageous when the thermoregulating/storing substance is a substance that works as a barrier of the heat propagation outside the lesion tissue area to be treated, the generated magnetic field being such that the ferromagnetic carrier concentrates the thermoregulating substance in an enveloping jacket of said lesion tissue area to be treated and the thermoregulating substance being provided with a vaporization or fusion temperature of 35 to 38° C.

The thermoregulating substance and/or the substance with ferromagnetic properties can be also contained in micro-bubbles or micro-balls and/or micro-bubbles or micro-balls can be the thermoregulating substance and/or the ferromagnetic substance.

A further variant provides that by means of injecting means a heat storing fluid is locally provided, particularly a substance having a predetermined temperature of the change of state as from liquid to gaseous and which temperature corresponds to the thermal treatment temperature of the lesion, which injector comes out at the output emitting the electromagnetic beam of the irradiating tip there being provided means for mechanically pushing said fluid.

The mechanical pushing of the heat storing fluid can be obtained in various ways, for example means for pushing said fluid can be composed of a direct conveying carrier composed of the natural lymphatic or vascular flow.

As an alternative to or in combination means pushing said fluid can be composed of a fluid jet there being provided on the tip of the probe or needle at least a nozzle supplying said jet or said jets.

Again as an alternative to or in combination means for pushing said fluid can be also composed of the mechanical pressure wave generated by a source of acustic waves particularly ultrasound ones. In this case low frequency ultrasound waves and with triangular or sawtooth pulse arrangement are advantageous.

A particular embodiment provides as the thermal storing fluid for transporting the thermal energy the vapour generated by heating the tissue by the electromagnetic beam coming from the irradiating tip.

Again according to a possible variant embodiment as regards the substance controlling the thermal diffusion it provides the use of substances that can modify the vascular and/or lymphatic circulation in the area corresponding to the lesion tissue. In such variant means for controlling the vascular or lymphatic circulation are advantageously composed of magnetorheological substances there being provided means generating localized magnetic fields operating magnetorheological substances to make agglomerates for locally preventing vascular and/or lymphatic flow generating a barrier to thermal diffusion by perfusion. A variant can be composed of means locally coagulating the blood in the lesion tissue area.

In this case an advantageous change of functions of thermal transfer from irradiating tip of the probe or needle in the direction moving away therefrom is obtained.

Referring particularly to FIG. 2, this figure shows the end of a needle or probe for thermal ablation corresponding to the tip irradiating a laser irradiation beam or ray. As it results from above mentioned documents the latter is generated by a laser source and it is transmitted through a thin optical fiber to the end of the needle or probe at which the irradiating tip is provided. In FIG. 2, the needle and the fiber are denoted together by reference number 1, while the arrow 2 denotes the direction of transmission of the laser irradiation ray or beam. The irradiating tip at which the irradiation comes out and by means of which it is directed against an area to be treated is denoted by 101.

In order to allow a thermal treatment corresponding to thermal specifications necessary for obtaining the action on lesion tissues and extending on an area, even a partial one, and as large as possible referring to the area wherein lesion tissues are localized or completely on said area and that is to the outermost borders of said area wherein lesion tissues are localized, the invention provides to associate at a certain distance from the irradiating tip a detector of the end treatment condition. In substance such detector denoted by 3 in FIG. 2 provides to detect physical or chemical parameters of the irradiated tissue that can change referring to temperature. Since the heating action changes depending on the distance from the irradiating tip, the distance and the threshold temperature detected depending on the physical or chemical parameter of the tissue change according to said physical or chemical parameter and correspondingly to the fact that in the highest heating area the temperature is under a predetermined allowable highest heating temperature.

In substance a transferring function of the heating action with reference to a change of a predetermined physical or chemical parameter of the treated tissue is defined and thus there is defined the greatest distance inside which the probe measuring said physical or chemical parameter can be positioned so that when the optimal treatment temperature is detected by said probe the tissue closest to the irradiating tip has not reached such temperatures overcoming a predetermined greatest temperature.

Providing the tip with a suitable diffuser for distributing the laser irradiation in the two directions along the axis of propagation, then it is possible to automatically determine the occurred treatment of an area that is substantially arranged in a symmetric way with respect to the irradiating tip in the direction of forward and backward propagation of the irradiation coming out from the irradiating tip of the probe or needle.

As the physical or chemical parameter depending on the temperature any physical or chemical parameters can be chosen. Firstly that depends on the kind of the tissue to be treated and on its physical or chemical characteristics.

Some typical physical or chemical parameters are electrical, thermal, acustic, optical, electrochemical parameters.

For example it is possible to measure the effect on the propagation of RF signals or a luminescence effect caused by the heating action of a tissue. PH or position changes or micromovements can be other effects that can be measured.

Once the physical or chemical parameter to be used for measuring the thermal transferring function has been determined so it is possible to find the type of measuring probe to be used.

In combination with the above it is also possible to provide to permeate the area to be treated with a substance having the known task of transferring the heating effect caused by the laser irradiation. In this case, the needle or probe are provided with means injecting said substance or the localized administration occurs by various individual administration devices.

For example if a lesion area such as the one delmited by the circle 4 in FIG. 3 is considered, once the detector 3 has detected that the temperature at it is such that it corresponds to the ideal treatment temperature, considering an isotropic distribution of the heating action then the volume in said circle would be all treated in the ideal provided way. The distance of the probe from the irradiating tip indicated by D and substantially corresponding to the radius of the circle is such that the temperature of the tissue provided directly contacting the irradiating tip 101 has not overcame the predetermined highest temperature when the probe has detected the ideal treatment temperature.

Thus it is possible to properly treat relatively large areas for ablating the lesion tissue without having the risk of an excessive heating of the tissue in the area immediately adjacent to the irradiating tip.

Characteristics of the probe according to FIGS. 2 and 3 can be provided in combination with means for controlling the diffusion of the heating effect according to the present invention in order to allow a signaling and control in an automatic way when possible the distribution of heat with reference to measurements carried out by temperature sensors.

1. Device for localized thermal ablation of lesion tissues, particularly tumoral tissues or the like, which device comprises:
a probe or needle intended to be positioned with the end tip at the lesion tissue or tumoral tissue area to be removed;
which probe or needle support at least a light guide as an elongated member like a thin wire or thread, one of the ends thereof is an end emitting heating electromagnetic energy and which light guide ends at said end of the probe or needle by a tip irradiating said electromagnetic energy, particularly as a laser light and the other end thereof is connected to a source generating the electromagnetic energy;
means for controlling the activation/deactivation of the source generating the electromagnetic energy.
and means for distributing the heating action on the lesion tissue area generated by the electromagnetic energy emitted by the irradiating tip inside a volume having a predetermined size, which means are composed of a fluid or a mixture or a combination of heat storing and/or conveying fluids or substances
Characterized in that means are provided for controlling the heat transfer from the fluid to the tissue by conveying and/or distributing a certain amount of fluid along diferent zones of the area to be treated and/or containing said fluid or fluids or said substance or substances inside different zones or the entire area to be treated or for conveying or containing said fluid or fluids or said substance or substances outside the area to be treated.

2. Device according to the present invention, characterized in that it comprises means for distributing the heat generated by the electromagnetic irradiation on distributing means.

3. Device according to claim 1 or 2, characterized in that it is provided in combination with means for injecting a thermoregulating substance, particularly a substance having a predetermined temperature of the change of state as for example from liquid to gaseous and/or from solid to liquid or vice versa and which temperature corresponds to the thermal treatment temperature of the lesion.

4. Device according to claim 3, characterized in that it further, comprises means for containing and/or retaining said substance inside a predetermined volume and/or outside it, particularly about a predetermined volume, which volume approximately coincides with the volume wherein the lesion tissue to be subjected to thermal ablation treatment is provided.

5. Device according to claim 4, characterized in that it is provided in combination with means injecting a thermoregulating substance having a ferromagnetic behaviour or associated to a conveying carrier composed of a substance with ferromagnetic properties, means for generating a localized magnetic field having such a spatial position and size to permeate only the lesion tissue area and/or to surround the lesion tissue area to be treated being provided, in order to distribute the thermoregulating substance in the volume corresponding approximately to the tissue area to be treated or around it along the surface envelopping said tissue area to be treated.

6. Device according to one or more of the preceding claims 3 to 5, characterized in that the thermoregulating substance is a substance that works as a barrier of the heat propagation outside the lesion tissue area to be treated, the generated magnetic field being such that the ferromagnetic carrier concentrates the thermoregulating substance in an envelopping jacket of said lesion tissue area to be treated and the thermoregulating substance being provided with a vaporization or fusion temperature of 35 to 38° C.

7. Device according to one or more of the preceiding claims 3 to 6, characterized in that the thermoregulating substance is a fluid with such a Curie temperature to have a ferromagnetic behaviour up to the treatment temperature and a diamagnetic behaviour over said temperature.

8. Device according to one or more of the preceding claims 3 to 7, characterized in that the thermoregulating substance and/or the substance with ferromagnetic properties are contained in micro-bubbles or micro-balls and/or micro-bubbles or micro-balls are the thermoregulating substance and/or the ferromagnetic substance.

9. Device according to one or more of the preceding claims, characterized in that it comprises in combination means injecting a heat storing fluid, particularly a substance having a predetermined temperature of the change of state such as from liquid to gaseous and which temperature corresponds to the thermal treatment temperature of the lesion, which injector comes out at the output emitting the electromagnetic beam of the irradiating tip there being provided means for pushing said fluid.

10. Device according to claims 9, characterized in that means for pushing said fluid are a direct conveying carrier composed of the vascular or natural lymphatic flux.

11. Device according to claim 9 or 10, characterized in that means for pushing said fluid are composed of a fluid jet there being provided on the tip of the probe or needle at least a nozzle supplying said jet or said jets.

12. Device according to claim 9 or 10, characterized in that means for pushing said fluid are the mechanical pressure wave generated by a source of acustic waves particularly ultrasound ones.

13. Device according to claim 12, characterized in that ultrasound waves have a low frequency and a triangular or sawtooth pulse arrangement.

14. Device according to one or more of the preceding claims 9 to 13, characterized in that the substance for transporting the thermal energy is the vapour generated by heating the tissue by the electromagnetic beam coming from the irradiating tip.

15. Device according to one or more of the preceding claims, characterized in that means for controlling the thermal diffusion are composed of means for controlling the vascular and/or lymphatic circulation in the area corresponding to the lesion tissue.

16. Device according to claim 15, characterized in that means for controlling the vascular or lymphatic circulation are composed of magnetorheological substances there being provided means generating localized magnetic fields operating magnetorheological substances to make agglomerates for locally preventing vascular and/or lymphatic flow generating a barrier to thermal diffusion by perfusion.

17. Device according to claim 15, characterized in that in combination it comprises means locally coagulating the blood.

18. Device according to one or more of the preceding claims, characterized in that it comprises at least a sensor measuring physical parameters of the lesion tissue depending on heating temperature thereof, which sensor is supported at a certain distance from the irradiating tip in a predetermined position with respect thereto and which sensor measures the change of said physical parameter of the lesion tissue comprised between said irradiating tip and the sensor and means for processing the measurement signal of the sensor which determine the heating temperature of said lesion tissue area on the basis of said measurement signal, as well as signaling means and/or possibly also automatic means for modulating the electromagnetic beam and/or automatic means for moving the irradiating tip operated on the basis of said measurement signal.

19. Device according to claim 18, characterized in that as an alternative or in combination the sensor can be of the electric, temperature, acustic, optical, laser, chemical, electrochemical, luminescence, RF wave change, pH, position, micro-movement, selective-tissue type.

20. Device according to claim 18 or 19, characterized in that for a predetermined type of lesion tissue in a predetermined anatomical district the correlation function between heating effect, heat diffusion and change in the physical parameter to be measured is determined, said function being sampled and stored in a table comparing and evaluating signals generated by the sensor measuring said physical parameter.

21. Method for localized thermal ablation of lesion tissues, particularly tumoral tissues or the like, which method comprises the following steps:
Generating an electromagnetic irradiation having a predetermined energy and frequency;
Irradiating locally and for a predetermined period of time, with said electromagnetic irradiation, a lesion tissure area or a portion thereof in order to increase the temperature of the lesion tissue of said area or portion thereof up to a predetermined value;
Characterized in that in combination it comprises steps controlling in an active way the distribution on the lesion tissue area of the heating action generated by the electromagnetic energy inside a volume having a predetermined size.

22. Method according to claim 21, characterized in that the control of the heat distribution is obtained by perfusing in the lesion tissue area a heat storing/thermoregulating substance, particularly a substance having a predetermined temperature of the change of state for example from liquid to gaseous and/or from solid to liquid or vice versa and which temperature corresponds to the temperature for thermally treating the lesion.

23. Method according to claim 21, characterized in that the control of the heat distribution is obtained by perfusing outside and about the lesion tissue area a heat termoregulating/storing substance, particularly a substance having a predetermined temperature of change of state for example from liquid to gaseous and/or from solid to liquid or vice-versa and which temperature corresponds to the average temperature of healthy tissue about the lesion tissue area.

24. Method according to claim 22 or 23, characterized in that the localized containing or retaining action inside the lesion tissue area or about the latter is obtained by ferromagnetic or storing substances having ferromagnetic properties or combined with carriers having ferromagnetic properties and by generating a localized magnetic field having such a spatial position and size to permeate only the lesion tissue area and/or to surround the lesion tissue area to be treated, in order to distribute the thermoregulating substance in the volume corresponding approximately to the tissue area to be treated or around it along the surface envelopping said tissue area to be treated.

25. Method according to claim 24, characterized in that the thermoregulating substance is a substance that works as a barrier of the heat propagation outside the lesion tissue area to be treated, the generated magnetic field being such that the ferromagnetic carrier concentrates the thermoregulating substance in an envelopping jacket of said lesion tissue area to be treated and the thermoregulating substance being provided with a vaporization or fusion temperature of 35 to 38° C.

26. Method according to one or more of the preceding claims 21 to 25, characterized in that it provides to heat a heat storing fluid by the electromagnetic irradiation, particularly a substance having a predetermined temperature of the change of state such as from liquid to gaseous and which temperature corresponds to the temperature for thermally treating the lesion, means for pushing said fluid in the lesion tissue area being provided.

27. Method according to claim 26, characterized in that pushing means are the vascular or natural lymphatic flux.

28. Method according to claim 26 or 27, characterized in that means for pushing said fluid are a fluid jet.

29. Method according to claim 26 or 27, characterized in that means for pushing said fluid are composed of the mechanical pressure wave generated by a source of acustic waves particularly ultrasound ones.

30. Method according to claim 29, characterized in that ultrasound waves have low frequency and a triangular or sawtooth pulse arrangement.

31. Method according to one or more of the preceding claims 26 to 30, characterized in that the substance for transporting the thermal energy is the vapour generated by heating the tissue by the electromagnetic beam.

32. Method according to one or more of the preceding claims 26 to 31, characterized the control of the thermal diffusion is carried out by controlling the vascular and/or lymphatic circulation in the area corresponding to the lesion tissue.

33. Method according to claim 32, characterized in that the control of the vascular or lymphatic circulation is obtained by activating magnetorheological substances by means of localized magnetic fields operating magnetorheological substances to make agglomerates for locally preventing vascular and/or lymphatic flow generating a barrier to thermal diffusion by perfusion.

34. Method according to claim 32, characterized in that it provides the localized coagulation of blood.
